(19) United States
(12) Patent Application Publication
(10) Pub. No.: US 2025/0244334 A1
(43) Pub. Date: Jul. 31, 2025

(54) AN IMPROVED METHOD FOR SEPARATING AND DETECTING OF FREE OR RESIDUAL POLYETHYLENE GLYCOL IN PEGYLATED PROTEIN MIXTURE

(71) Applicant: KASHIV BIOSCIENCES, LLC, Piscataway, NJ (US)

(72) Inventors: Chintan Kumar SHAH, Ahmedabad (IN); Namrata Ajit MORE, Ahmedabad (IN); Shalini SHARMA, Piscataway, NJ (US)

(21) Appl. No.: 18/853,229

(22) PCT Filed: Mar. 30, 2023
(86) PCT No.: PCT/IB2023/053182

§ 371 Date: Oct. 1, 2024

(30) Foreign Application Priority Data
Apr. 1, 2022 (IN) 202221019961

(51) Int. Cl. G01N 33/68; B01D 15/32; B01D 15/34; B01D 15/42; G01N 30/02; G01N 30/46; G01N 30/64; G01N 30/88

(52) U.S. Cl. CPC G01N 33/6803; B01D 15/327; B01D 15/34; B01D 15/424; G01N 30/463; G01N 30/64; G01N 30/88; G01N 2030/027; G01N 2030/8831; G01N 2440/28

(57) ABSTRACT

The present invention relates to an improved method for separating and detecting of free or residual polyethylene glycol (PEG) in pegylated protein mixture. In particular, the method improves sensitivity, effective separation, and robustness of the quantification of free or residual PEG and related substance present in pegylated protein mixture which obtained as drug substance or drug product.

### FIELD OF INVENTION

The present invention relates to an improved method for separating and detecting of free or residual polyethylene glycol (PEG) in pegylated protein mixture. In particular, the method improves sensitivity, effective separation, and robustness of the quantification of free or residual PEG and related substance present in pegylated protein mixture which obtained as drug substance or drug product.

### BACKGROUND

PEG is a water-soluble, nontoxic, non-antigenic, biocompatible polymer that has been approved by the Food and Drug Administration for human intravenous, oral, and dermal applications. Attachment of PEG (PEGylation) to drugs, peptides, proteins, nanoparticles, micelles, and liposomes is a mature technology for enhancing the bioavailability, stability, safety, and efficacy of a wide range of therapeutic agents. Therefore, PEGylated molecules are increasingly employed as mainstream therapeutic and diagnostic agents, which in turn have created great demand for methods facilitating the qualitative and quantitative analysis of PEG-derivatized molecules for both drug development and clinical applications.

High performance liquid chromatography (HPLC) is nowadays one of the most widespread techniques in analytical laboratories, and modern pharmaceutical analysis and quality control would not be thinkable without. Due to its wide field of applications, its sensitivity, the wide range of linearity and the compatibility with gradient elution, the most common detection technique for HPLC is UV/vis spectrophotometry.

However, UV/vis and also fluorescence detection come to their limits for the actual detection and quantification of PEG and PEG reagents when the analyte molecules are lacking a suitable chromophor or fluorophore and the sensitivity and accuracy of those quantifications are still lacking.

It has been observed that during the analysis of PEGylated protein samples, proteins and other components such as excipients in the buffer solution interfere with the PEG molecule while separating from each other which leads to the inaccurate results.

Therefore, a need exists to provide a more reliable method for sensitive quantification and/or detection and separation of PEG and PEG reagents. The present invention overcome the above problem by employing specific chromatography columns in a specific manner/combinations in conjunction with a suitable detector.

### SUMMARY OF INVENTION

The present invention provides a method for separating and detecting free or residual polyethylene glycol in a pegylated protein mixture. Methods disclosed herein capable of effectively separating and detecting free polyethylene glycol molecule in the pegylated protein mixture.

Methods disclosed herein comprises chromatographic steps in a series followed by Charge Aerosol Detector (CAD).

In one embodiment, the process disclosed herein provides a method of separating and detecting free PEG in pegylated protein mixture using at least two chromatography steps which are connected in a series.

In one embodiment, the process disclosed herein provides a method of separating and detecting free PEG in pegylated protein mixture using two chromatography comprising of Reverse Phase-High Performance Liquid Chromatography (RP-HPLC) and Size Exclusion Chromatography (SEC).

In one embodiment, the process disclosed herein provides a method of separating and detecting free PEG in pegylated protein mixture using two chromatography comprising of Reverse Phase-High Performance Liquid Chromatography (RP-HPLC) and Size Exclusion Chromatography (SEC). The free PEG is detected or quantified through suitable detector.

In one embodiment, the process disclosed herein provides a method of separating and detecting free PEG in pegylated protein mixture using two chromatography comprising of Reverse Phase-High Performance Liquid Chromatography (RP-HPLC) and Size Exclusion Chromatography (SEC). The free PEG is detected or quantified through charge aerosol detector.

In another embodiment, the process disclosed herein provides a method of separating and detecting free PEG and excipients in pegylated protein mixture using two chromatography comprising of Reverse Phase-High Performance Liquid Chromatography (RP-HPLC) and Size Exclusion Chromatography (SEC). The free PEG is detected or quantified through suitable detector.

In one embodiment, the method provides separation of free or residual polyethylene glycol from pegylated protein mixture comprising:
a) obtaining a protein mixture comprising a pegylated protein and at least free or residual polyethylene glycol;
b) subjecting the protein mixture to RP-HPLC column to obtain first treated protein mixture;
c) subjecting the first treated protein mixture to size exclusion chromatography column to obtain quantifiable protein mixture; and
d) separation and detection of free PEG from the quantifiable protein mixture.

In one embodiment, the method for detecting or quantifying free or residual polyethylene glycol in pegylated protein mixture comprising:
a) obtaining a protein mixture comprising a pegylated protein and at least free or residual polyethylene glycol;
b) subjecting the protein mixture to RP-HPLC column to obtain first treated protein mixture;
c) subjecting the first treated protein mixture to size exclusion chromatography column to obtain quantifiable protein mixture; and
d) quantification and detection of free PEG in the quantifiable protein mixture.

In one embodiment, the method provides separation of free or residual polyethylene glycol from pegylated protein mixture comprising:
a) obtaining a protein mixture comprising a pegylated protein and at least free or residual polyethylene glycol;
b) subjecting the protein mixture to RP-HPLC column to obtain first treated protein mixture in flow-through mode and second treated protein mixture binds to RP-HPLC;
c) subjecting the first treated protein mixture to size exclusion chromatography column to obtain quantifiable protein mixture;
d) eluting the second treated protein mixture from the RP-HPLC; and
e) separating and detection of free PEG in the quantifiable protein mixture.

In one embodiment, the method for detecting or quantifying free or residual polyethylene glycol in pegylated protein mixture comprising:
a) obtaining a protein mixture comprising a pegylated protein and at least free or residual polyethylene glycol;
b) subjecting the protein mixture to RP-HPLC column to obtain first treated protein mixture in flow-through mode and second treated protein mixture binds to RP-HPLC;
c) subjecting the first treated protein mixture to size exclusion chromatography column to obtain quantifiable protein mixture;
d) eluting the second treated protein mixture from the RP-HPLC; and
e) quantification and detection of free PEG in the quantifiable protein mixture.

In one embodiment, the method for detecting or quantifying pegylated protein in pegylated protein mixture comprising:
a) obtaining a protein mixture comprising a pegylated protein and at least free or residual polyethylene glycol;
b) subjecting the protein mixture to RP-HPLC column to obtain first treated protein mixture in flow through mode and second treated protein mixture binds to RP-HPLC;
c) subjecting the first treated protein mixture to size exclusion chromatography column to obtain quantifiable protein mixture;
d) eluting the second treated protein mixture from the RP-HPLC;
e) quantification and detection of free PEG in the quantifiable protein mixture; and
f) quantification and detection of pegylated protein in the second protein mixture.

In one embodiment, the method for separation & detection of pegylated protein in pegylated protein mixture comprising:
a) obtaining a protein mixture comprising a pegylated protein and at least free or residual polyethylene glycol;
b) subjecting the protein mixture to RP-HPLC column to obtain first treated protein mixture in flow through mode and second treated protein mixture binds to RP-HPLC;
c) subjecting the first treated protein mixture to size exclusion chromatography column to obtain quantifiable protein mixture;
d) eluting the second treated protein mixture from the RP-HPLC;
e) performing separation and detection of free PEG in the quantifiable protein mixture; and
f) performing separation and detection of pegylated protein in the second protein mixture.

In one embodiment, the method for detecting or quantifying pegylated protein, free or residual polyethylene glycol in pegylated protein mixture comprising:
a) obtaining a protein mixture comprising a pegylated protein and at least free or residual polyethylene glycol;
b) subjecting the protein mixture to RP-HPLC column to obtain first treated protein mixture in flow through mode and second treated protein mixture binds to RP-HPLC;
c) subjecting the first treated protein mixture to size exclusion chromatography column to obtain quantifiable protein mixture;
d) eluting the second treated protein mixture from the RP-HPLC;
e) quantification and detection of free PEG in the quantifiable protein mixture; and
f) quantification and detection of pegylated protein in the second protein mixture.

In one embodiment, the method for detecting or quantifying free or residual polyethylene glycol in pegylated protein mixture comprising:
(a) obtaining a protein mixture comprising a pegylated protein and at least free or residual polyethylene glycol;
(b) subjecting the protein mixture to at least two chromatography steps; wherein the first chromatography step is capable to bind the protein of interest and second chromatography step is capable to separate based on molecular weight or size of the analyte.
(c) eluting the protein mixture; and
(d) quantification and detection of free PEG and PEGylated protein in the protein mixture by a suitable detector.

In an embodiment, the PEGylated protein in the protein mixture is PEG-GCSF.

In an embodiment, the chromatography steps comprise two chromatography steps.

In an embodiment, the two chromatography steps are RP-HPLC and SEC-HPLC.

In an embodiment, the two chromatography steps are RP-HPLC followed by SEC-HPLC connected in a series or performed as an individual chromatography.

In an embodiment, elution of protein mixture as a “first protein mixture” eluted in flow through mode and as “second protein mixture” eluted in bind eluate mode from RP-HPLC.

In an embodiment, free or residual polyethylene glycol is subjected to SEC-HPLC followed by PEGylated protein.

In an embodiment, detection or quantification of free or residual PEG and PEGylated protein in the protein mixture by Charged Aerosol Detector.

In an embodiment, the method for detecting or quantifying free or residual polyethylene glycol and PEG-GCSF in pegylated protein mixture comprising two chromatography steps (a) RP-HPLC, and (b) SEC-HPLC followed by Charged Aerosol Detector.

### BRIEF DESCRIPTION OF FIGURE

FIG. 1: depicts the chromatogram of single SEC column, a) PEG standard 10 ppm, b) PEG standard 1000 ppm, c) Sample (protein) peak is overlapping on PEG peak (Sample Diluted 1:10 with water)

FIG. 2: depicts the chromatogram of single RP-HPLC column, a) PEG standard 100 ppm, b) Buffer peak is co-eluting with PEG peak (Sample diluted with water 1:10)

FIG. 3: depicts the chromatogram of two column in a series (RP-HPLC followed by SEC), a) Blank, b) Separation of free PEG or residual PEG from Pegylated protein and buffer.

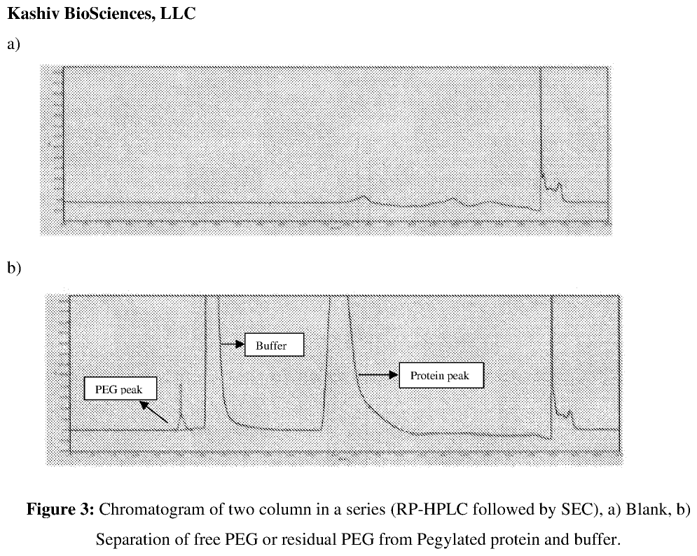

FIG. 4: depicts the chromatogram of (a) Blank solution, (b) mPEG-PAL Standard Solution (0.05 mg/mL), (c) Separation of mPEG-PAL Standard, buffer and protein from the PEGylated GCSF (DS) Sample.

FIG. 5: depicts the chromatogram of CAD showing (a) Overlay of Diluent (Black), DP formulation buffer (Red), GCSF (DS)-Green, (b) Peak profile of mPEG-PAL (0.05 mg/mL), (c) Overlay of PEGylated GCSF (DP), Neulasta, PEGylated GCSF (DS) and Primary reference standard (PRS).

FIG. 6: depicts the chromatogram of mPEG-PAL accuracy regression analysis.

### DETAILED DESCRIPTION OF THE INVENTION

The present invention provides a method for effectively quantifying free polyethylene glycol molecule in the pegylated protein mixture.

In certain embodiment, the invention provides the quantification of PEGylated protein in the protein mixture.

As used herein the term “protein mixture” refers to a mixture of pegylated protein, free PEG or residual PEG. In certain embodiment, the protein mixture comprises a pegylated protein which is protein of interest, free or residual PEG which is an impurity derived from Pegylated protein. In certain embodiment, the protein mixture further comprises an excipient which are selected from formulation component or excipients and derivative thereof.

As used herein the term “First treated protein mixture” refers to protein mixture obtained from at least one chromatography column preferably from RP-HPLC. First treated protein mixture comprises a substantial amount of free PEG or residual PEG. In certain embodiment, the first treated protein mixture comprises a substantially amount of excipients. In an embodiment, the first treated protein mixture comprises a substantially amount of free PEG or residual PEG and excipients. It is important to note that the free PEG or residual PEG and excipients elutes from the RP-HPLC in flow through mode.

As used herein the term “quantifiable protein mixture” refers to a protein mixture eluted from at least one chromatography column and capable to be used for detection or quantification of free PEG or residual PEG. In certain embodiment, the protein mixture eluted from at least one chromatography column and capable to be used for detection or quantification of free PEG or residual PEG and excipients. Quantifiable protein mixture can be subjected to any suitable detector known in the art preferably present invention employs CAD. In an embodiment, the quantifiable protein mixture is obtained from chromatography column selected from RP-HPLC and Size Exclusion Chromatography. Any skilled person can use the knowledge of the present invention to either employ both column in continuous mode or in standalone which will be considered under the scope of the present invention.

As used herein the term “second treated protein mixture” refers to a protein mixture obtained from at least one chromatography column preferably from RP-HPLC. The second treated protein mixture comprises a predominately pegylated protein in eluted from RP-HPLC as Pegylated protein binds to the RP-HPLC and therefore eluted in bind elute mode. In certain embodiment, the second treated protein mixture elutes after the elution of first treated protein mixture.

As used herein the term “substantially” refers to presence of free PEG or residual PEG and excipients more than 30% in treated protein mixture. In certain embodiment, the free PEG or residual PEG and excipients are present more than 40%, 50%, 60%, 70%, 80%, 85%, 90%, 91%, 92%, 93%, 94%, 95%, 96%, 97%, 98%, 99%, 100% in treated protein mixture.

In an embodiment, the present invention employs at least two chromatography columns. Second treated protein mixture elutes from the first chromatography column when the first treated protein mixture load onto the second column.

In certain embodiment, second treated protein mixture elutes from the first chromatography column when the first treated protein mixture passes through the second column.

In certain embodiment, second treated protein mixture elutes from the first chromatography column when the first treated protein mixture elutes from the second column.

In certain embodiment, second treated protein mixture elutes from the first chromatography column when the first treated protein mixture elutes from the second column to form quantifiable protein mixture.

In an embodiment, the second treated protein mixture is further subjected to second chromatography column and thereafter used for detection or quantification of pegylated protein.

In an embodiment, the second treated protein mixture elutes from RP-HPLC and further subjected to Size Exclusion Chromatography and thereafter pass-through detector CAD for detection or quantification of pegylated protein.

In certain embodiment, the second treated protein mixture is not further subjected to second chromatography column and can be directly used for detection or quantification of pegylated protein. In an embodiment, the second treated protein mixture elutes from RP-HPLC and thereafter pass-through detector CAD for detection or quantification of pegylated protein.

As used herein the term “GCSF” or “Granulocyte colony stimulating factor” refers to a hematopoietic growth factor that stimulates the production of white blood cells (neutrophils). It involves in the development and functional activation of hematopoietic elements. It also stimulates neutrophil migration. GCSF has a molecular weight of about 18-19 kDa. In certain embodiment is GCSF or PEG-GCSF is similar to filgrastim or PEG-filgrastim.

As used herein the term “free PEG” or “residual PEG” or “Polyethylene glycol” refers to a water-soluble, non-toxic, non-antigenic, biocompatible polymer with molecular weight of about 20 kDa that has been approved by the Food and Drug Administration (FDA) for human intravenous, oral, and dermal applications.

As used herein the term “mPEG-PAL” or “monomethoxy polyethylene glycol propionaldehyde” refers to a standard free PEG molecule.

As used herein the term “Neulasta” refers to the Primary reference standard which is pegylated GCSF.

As used herein the term “Pegylated protein” refers to protein conjugated with PEG. In certain embodiment, the protein is selected from antibody or fragment thereof, cytokines like GCSF, IFN, growth factors and other recombinant protein suitable for pegylation. In certain embodiment, the Fab fragment of antibody is attached with PEG. In certain embodiment, the PEG molecular weight is selected from 5 kDa, 10 kDa, 15 kDa, 18 kDa, 20 kDa, 25 kDa, 30 kDa, 35 kDa and 40 kDa.

As used herein the term “PEGylated GCSF or PEG-GCSF” refers to a protein where the PEG moiety is covalently conjugated to the N-terminus of GCSF (Granulocyte colony stimulating factor) which is employed for chemotherapy-induced neutropenia in patients due to its longer half-life.

As used herein the term “excipients” refers to the substances other than the active pharmaceutical ingredient (API) that have been appropriately evaluated for safety and are intentionally included in a drug delivery system. Excipients have different roles in a formulation. It protects, supports, or enhance stability, bioavailability, or patient acceptability and assists in maintaining the integrity of the drug product during storage.

As used herein the term “RP-HPLC” or “Reverse Phase-High performance liquid chromatography refers to a chromatography technique, which is used to separate molecules based upon the reversible adsorption/desorption of solute molecules with changing levels of hydrophobicity to a hydrophobic stationary phase.

As used herein the term “SEC” or “Size exclusion chromatography” refers to a chromatography technique, which is used to separate molecules based on their size by filtration through a gel.

As used herein the term “CAD” or “Charged aerosol detector” also known as “Corona”, are well known as universal detectors for use in conjunction with liquid chromatography. In short, their action is based on nebulizing into droplets the eluent containing the analytes when it exits the column, subsequently evaporating the solvent to form particles, charging the particles in a reaction chamber by collision with ionized nitrogen, which is formed when nitrogen is passed over a corona wire, and measuring the particle charge by a sensitive electrometer. This generates a signal directly proportional to the quantity of analyte present. Any analyte, as long as it forms a particle, can be measured by charged aerosol detection, regardless of its chemical structure.

As used herein the term “flow through mode” or “flow through” refers to a purification process wherein the molecule is not bound to a chromatography resin but is instead obtained in the unbound or “flow-through” during loading or post loading washes of a chromatography support.

As used herein the term “eluting” or “elution” or “bind elute mode” refers to the chromatographic methods where in a protein molecule is bound to a chromatography resin when loaded onto a resin column and is subsequently eluted using an elution buffer.

As used herein the term “column” or “resin” or “chromatographic resin” refers to the media used to capture and polish proteins and other biomolecules using a stationary phase.

As used herein the term “column” or “resin” or “chromatographic resin or chromatographic column” are interchangeable.

As used herein the term “drug substance” refers to the active ingredient or active pharmaceutical ingredient which is used to make the drug product. It is a pure material (without excipients) which exerts pharmacological action on the body.

As used herein the term “drug product” refers to the finished product of any drug that is available in the market and is ready to use.

As used herein the term “comprises” or “comprising” is used in the present description, it does not exclude other elements or steps. For the purpose of the present invention, the term “consisting of” is considered to be an optional embodiment of the term “comprising of”. If hereinafter a group is defined to comprise at least a certain number of embodiments, this is also to be understood to disclose a group which optionally consists of these embodiments.

As used herein the term “about” is intended to refer to ranges of approximately 10-20% greater than or less than the referred value. In certain circumstances, one of skill in the art will recognise that, due to the nature of referenced value, the term “about” can mean more or less than a 10-20% deviation from that value.

As used herein the term “gradient” or “gradient elution”, where the concentration of solvent A and solvent B mobile phase. In certain embodiment the solvent A and solvent B is maintained in 1:1 ratio. In certain embodiment solvent B is gradually increased in comparison to solvent A. In certain embodiment solvent B is gradually decreased in comparison to solvent A.

As used herein the term “mobile phase A” refers to a buffer solution prepared by ammonium acetate.

As used herein the term “mobile phase B” refers to a solution of acetonitrile.

As used herein the term “Column 1” refers to a Reverse Phase-High performance liquid chromatography (RP-HPLC) column.

As used herein the term “Column 2” refers to a Size exclusion chromatography (SEC) column.

As used herein the term “Negative Control” refers to a sample (diluent) completely free from free-PEG or protein molecule or excipients.

In one embodiment, the method provides separation of free or residual polyethylene glycol from pegylated protein mixture comprising:
a) obtaining a protein mixture comprising a pegylated protein and at least free or residual polyethylene glycol;
b) subjecting the protein mixture to RP-HPLC column to obtain first treated protein mixture;
c) subjecting the first treated protein mixture to size exclusion chromatography column to obtain quantifiable protein mixture; and
d) separation and detection of free PEG from the quantifiable protein mixture.

In one embodiment, the method for detecting or quantifying free or residual polyethylene glycol in pegylated protein mixture comprising:
a) obtaining a protein mixture comprising a pegylated protein and at least free or residual polyethylene glycol;
b) subjecting the protein mixture to RP-HPLC column to obtain first treated protein mixture;
c) subjecting the first treated protein mixture to size exclusion chromatography column to obtain quantifiable protein mixture; and
d) quantification and detection of free PEG in the quantifiable protein mixture.

In one embodiment, the method provides separation of free or residual polyethylene glycol from pegylated protein mixture comprising:
a) obtaining a protein mixture comprising a pegylated protein and at least free or residual polyethylene glycol;
b) subjecting the protein mixture to RP-HPLC column to obtain first treated protein mixture in flow-through mode and second treated protein mixture binds to RP-HPLC;
c) subjecting the first treated protein mixture to size exclusion chromatography column to obtain quantifiable protein mixture;
d) eluting the second treated protein mixture from the RP-HPLC; and
e) separating and detection of free PEG in the quantifiable protein mixture.

In one aspect of such embodiment, the protein mixture comprising a pegylated protein, free PEG or residual PEG & excipients.

In an embodiment, the pegylated protein is selected from pegademase bovine (PEG-ADA), pegaspargase, pegfilgrastim (PEG-GCSF), peg-interferons (PEG-IFN), pegvisomant, pegaptanib, certolizumab pegol, PEGylated recombinant human growth hormone (PEG-rhGH), PEGylated epoetin B, PEGylated recombinant antihemophilic factor, I PEGylated recombinant human erythropoietin (PEG-rHuEPO), PEG-uricase, PEGylated antitumor necrosis factor (PEG-rHuTNF-alpha) and certolizumab pegol.

In an embodiment, the PEGylated protein is PEGylated Granulocyte Colony Stimulating Factor (PEG-GCSF).

In one embodiment, the method for detecting or quantifying free or residual polyethylene glycol in pegylated protein mixture comprising:
a) obtaining a protein mixture comprising a pegylated protein and at least free or residual polyethylene glycol;
b) subjecting the protein mixture to RP-HPLC column to obtain first treated protein mixture;
c) subjecting the first treated protein mixture to size exclusion chromatography column to obtain quantifiable protein mixture; and
d) quantification and detection of free PEG in the quantifiable protein mixture.

In one aspect of such embodiment, the chromatographic columns are selected from Size exclusion chromatography (SEC), Reverse Phase-High performance liquid chromatography (RP-HPLC), RP-HPLC followed by SEC in a series.

In one embodiment, the chromatographic columns are RP-HPLC followed by SEC in a series.

In one embodiment, the method for detecting or quantifying pegylated protein, free or residual polyethylene glycol in pegylated protein mixture comprising:
a) obtaining a protein mixture comprising a pegylated protein and at least free or residual polyethylene glycol;
b) subjecting the protein mixture to RP-HPLC column to obtain first treated protein mixture in flow through mode and second treated protein mixture binds to RP-HPLC;
c) subjecting the first treated protein mixture to size exclusion chromatography column to obtain quantifiable protein mixture;
d) eluting the second treated protein mixture from the RP-HPLC;
e) quantification and detection of free PEG in the quantifiable protein mixture; and
f) quantification and detection of pegylated protein in the second protein mixture

In one aspect of such embodiment, the detector for detection & quantification of protein mixture selected from Evaporative light scattering detectors (ELSD), Condensation nucleation light scattering detectors (CNLSD) & Charge aerosol detector (CAD).

In an embodiment, the detector is Charge aerosol detector (CAD).

In an embodiment, the PEGylated protein mixtures are eluted from RP-HPLC column by using elution buffer mobile phase A and mobile phase B comprising acetonitrile and ammonium acetate.

In one aspect of such embodiment, the gradient conditions are selected from about 30%, about 40%, about 50%, about 60%, about 70%, about 80%, about 90% and about 100% of mobile phase B.

In an embodiment, the gradient conditions were selected from about 50% to about 90% of mobile phase B.

In one aspect of such embodiment, the sample temperature was selected from about 2° C. to about 10° C.

In an embodiment, the sample temperature selected is about 5° C.

In one aspect of such embodiment, the Evaporator temperatures are selected from about 25° C. to about 45° C.

In an embodiment, the Evaporator temperature selected is about 35° C.

In one embodiment, the method for separation & detection of pegylated protein in pegylated protein mixture comprising:
a) obtaining a protein mixture comprising a pegylated protein and at least free or residual polyethylene glycol;
b) subjecting the protein mixture to RP-HPLC column to obtain first treated protein mixture in flow through mode and second treated protein mixture binds to RP-HPLC;
c) subjecting the first treated protein mixture to size exclusion chromatography column to obtain quantifiable protein mixture;
d) eluting the second treated protein mixture from the RP-HPLC;
e) performing separation and detection of free PEG in the quantifiable protein mixture; and
f) performing separation and detection of pegylated protein in the second protein mixture.

In one embodiment, the method for detecting or quantifying free or residual polyethylene glycol in pegylated protein mixture comprising:
(a) obtaining a protein mixture comprising a pegylated protein and at least free or residual polyethylene glycol;
(b) subjecting the protein mixture to at least two chromatography steps; wherein the first chromatography step is capable to bind the protein of interest and second chromatography step is capable to separate based on molecular weight or size of the analyte;
(c) eluting the protein mixture; and
(d) quantification and detection of free PEG and PEGylated protein in the protein mixture by a suitable detector.

In an embodiment, the PEGylated protein in the protein mixture is PEG-GCSF.

In an embodiment, the chromatography steps comprise two chromatography steps.

In an embodiment, the two chromatography steps are RP-HPLC and SEC-HPLC.

In an embodiment, the two chromatography steps are RP-HPLC followed by SEC-HPLC connected in a series or performed as an individual chromatography.

In an embodiment, elution of protein mixture as a “first protein mixture” eluted in flow through mode and as “second protein mixture” eluted in bind eluate mode from RP-HPLC.

In an embodiment, free or residual polyethylene glycol is subjected to SEC-HPLC followed by PEGylated protein.

In an embodiment, detection or quantification of free or residual PEG and PEGylated protein in the protein mixture by Charged Aerosol Detector.

In an embodiment, the method for detecting or quantifying free or residual polyethylene glycol and PEG-GCSF in pegylated protein mixture comprising two chromatography steps (a) RP-HPLC, and (b) SEC-HPLC followed by Charged Aerosol Detector.

### Example 1: Separation and Quantification of PEG in PEGylated GCSF Samples by Single Column Size Exclusion Chromatography (SEC)

The present example is performed to quantify the free or residual polyethylene glycol in PEGylated GCSF samples which is critical intermediate in the PEGylation reaction, expressed as mg/mL in PEGylated GCSF samples by using single column SEC with Charged aerosol detector (CAD).

### Preparations:

1. Diluent: Water 100% (LC/MS grade) as diluent.
2. Dilute acetic acid: Acetic acid diluted 1:1 with water (LC/MS grade).
3. Mobile phase A: 7.7153 gm of ammonium acetate was dissolved in 1000 ml of water (LC/MS grade) by sonication and pH was adjusted to 5.0 with 50% diluted glacial acetic acid and then filtered through 0.22 μm membrane filter and marked as mobile phase A.
4. Mobile phase B: 1000 mL 100% acetonitrile (LC/MS grade) was measured in glass bottle and marked as mobile phase B.
5. PEG standard solution preparation (1000 ppm): 20.39 mg of (Polyethylene glycol 20KD) PEG was weighed & transferred in 20 ml volumetric flask, added 10 ml of diluent, and dissolved by approximately 2 minutes sonication, then made up to the mark and mixed well.
6. PEG standard solution preparation (100 ppm): 1 ml of standard stock solution was pipetted and transferred in 10.0 ml volumetric flask then made up to the mark with diluent and mixed well.
7. PEG standard solution preparation (10 ppm): 1 ml of 100 ppm standard stock solution was pipetted and transferred in 10.0 ml volumetric flask then made up to the mark with diluent and mixed well.
8. Sample preparation: 100 μl of sample and transferred in 1 ml HPLC vial containing 900 μl of diluent and mixed well.

TABLE 1
HPLC Method Parameters:
Instrument Parameter Value

| Column description | Acclaim SEC-1000A°, 7 μm, 7.8 × 300 mm | |
|---|---|---|
| Flow rate | 1.0 | mL/min |
| Column oven temperature | 25° | C. |
| Auto Sampler | 20° | C. |
| Injection volume | 100 | μL |
| Run time | 20 | minutes |

| | |
|---|---|
| Detector | CAD |
| Needle wash | Mobile phase A:Mobile phase B (50:50 V/V) |
| Elution Isocratic | Mobile Phase A: 75% |
| | Mobile phase B: 25% |

TABLE 2
Charged aerosol Detector Parameter:
Instrument Parameter Value

| Evaporator temp | Low | |
|---|---|---|
| Wait ready | ±5.0K | |
| Peak width | 0.1 | min. |
| Data collection rate | 10.0 | Hz |
| Filter | 5.0 | Sec. |

| | |
|---|---|
| Power function | 1.0 |

TABLE 3
Gradient Program:
Gradient Conditions

| Time (min.) | % Mobile phase B |
|---|---|
| 0.0 | 25 |
| 20.0 | 25 |

### Results:

| Sample | Retention time (min) |
|---|---|
| RT of PEG peak in 10 ppm standard | 8.063 |
| RT of PEG peak in 1000 ppm standard | 8.022 |
| RT of PEG peak in sample | 7.938 |

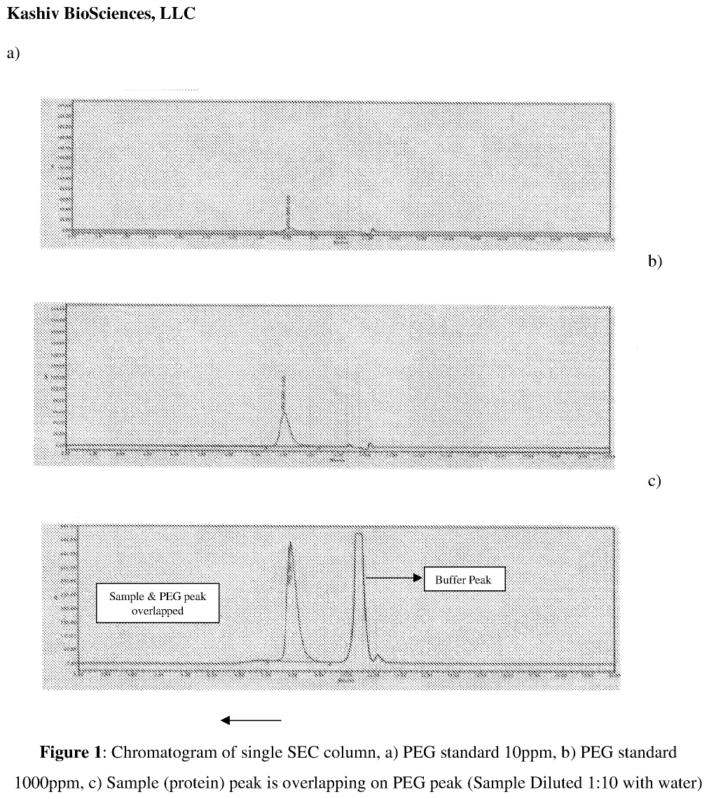

In Acclaim SEC 1000A° single column PEG peak shape was good with tailing 1.4, but sample (protein) peak is overlapping on PEG peak as shown in FIG. 1.

### Example 2: Separation and Quantification of PEG in PEGylated GCSF Samples by Single Column Reverse Phase-High Performance Liquid Chromatography (RP-HPLC)

The present example is performed to quantify the free or residual polyethylene glycol in PEGylated GCSF samples which is critical intermediate in the PEGylation reaction, expressed as mg/mL in PEGylated GCSF samples by using single column RP-HPLC with Charged aerosol detector (CAD).

### Preparations:

1. Diluent: Water 100% (LC/MS grade) as diluent.
2. Dilute acetic acid: Acetic acid diluted 1:1 with water (LC/MS grade).
3. Mobile phase A: 7.7153 gm of ammonium acetate was dissolved in 1000 ml of water (LC/MS grade) by sonication and pH was adjusted to 5.0 with 50% diluted glacial acetic acid and then filtered through 0.22 μm membrane filter and marked as mobile phase A.
4. Mobile phase B: 1000 mL 100% acetonitrile (LC/MS grade) was measured in glass bottle and marked as mobile phase B.
5. PEG standard solution preparation (1000 ppm): 20.39 mg of (Polyethylene glycol 20KD) PEG was weighed & transferred in 20 ml volumetric flask, added 10 ml of diluent, and dissolved by approximately 2 minutes sonication, then made up to the mark and mixed well.
6. PEG standard solution preparation (100 ppm): 1 ml of standard stock solution was pipetted and transferred in 10.0 ml volumetric flask then made up to the mark with diluent and mixed well.
7. PEG standard solution preparation (10 ppm): 1 ml of 100 ppm standard stock solution was pipetted and transferred in 10.0 ml volumetric flask then made up to the mark with diluent and mixed well.
8. Sample preparation: 100 μl of sample and transferred in 1 ml HPLC vial containing 900 μl of diluent and mixed well.

TABLE 4
HPLC Method Parameters:
Instrument Parameter Value

| Column description | Acclaim C18 300 (150*4.6) 3 μm | |
|---|---|---|
| Flow rate | 1.0 | mL/min |
| Column oven temperature | 25° | C. |
| Auto Sampler | 20° | C. |
| Injection volume | 20 | μL |
| Run time | 20 | minutes |

| | |
|---|---|
| Detector | CAD |
| Needle wash | Mobile phase A:Mobile phase B (50:50 V/V) |

TABLE 5
Charged Aerosol Detector Parameter:
Instrument Parameter Value

| Evaporator temp | Low | |
|---|---|---|
| Wait ready | ±5.0K | |
| Peak width | 0.1 | min. |
| Data collection rate | 10.0 | Hz |
| Filter | 5.0 | Sec. |

| | |
|---|---|
| Power function | 1.0 |

TABLE 6
Gradient Program:
Gradient Conditions

| Time (min.) | % Mobile phase B |
|---|---|
| 0.0 | 50 |
| 5.0 | 50 |
| 10.0 | 90 |
| 22.0 | 90 |
| 23.0 | 50 |
| 30.0 | 50 |

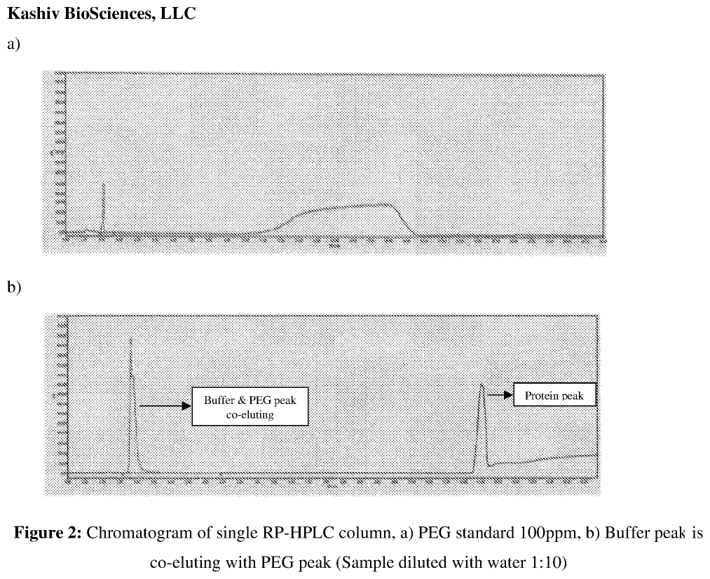

Results: PEG peak is co-eluting with Buffer peak as shown in FIG. 2. PEG retention time −2.073, tailing −0.9, and Buffer −1.807

### Example 3: Separation and Quantification of Free PEG or mPEG-PAL (Monomethoxy Propionaldehyde) in PEGylated GCSF Samples by Using Two Column RP-HPLC Followed by SEC with Charged Aerosol Detector (CAD)

The present example is performed to quantify the free or residual polyethylene glycol in PEGylated GCSF samples which is critical intermediate in the PEGylation reaction, expressed as mg/mL in PEGylated GCSF samples with two chromatography columns (RP-HPLC followed by SEC).

### Preparations:

1. Samples: Diluted test sample 1:1 with Acetonitrile (LC-MS grade). i.e., 50 μl of test sample diluted with 50 μl of Acetonitrile.
2. Mobile phase A: 7.7153 gm of ammonium acetate was dissolved in 1000 ml of water (LC/MS grade) by sonication and pH was adjusted to 5.0 with 50% diluted glacial acetic acid and then filtered through 0.22 μm membrane filter and marked as mobile phase A.
3. Mobile phase B: 1000 mL 100% acetonitrile (LC/MS grade) was measured in glass bottle and marked as mobile phase B.
4. Stock and working solution preparation of Standard (mPEG-PAL): 50 mg mPEG-PAL was weighed and transferred to 50 mL volumetric flask. Volume is marked with water and labelled it as Stock solution (1.0 mg/mL). 0.5 mL of stock solution was transferred to 10 mL of volumetric flask and marked with water. This solution was labelled as working solution.

TABLE 7
HPLC Method Parameters:
Instrument Parameter Value

| | |
|---|---|
| Column 1 | Acclaim 300A°, C18, 3 μm, 4.6 × |
| | 150 mm |
| Column 2 | Acclaim SEC-1000A°, 7 μm, 7.8 × |
| | 300 mm |
| Flow rate | 1.0 mL/min |
| Column oven | Ambient |
| temperature | |
| Needle wash Mobile | Mobile phase B (50:50 V/V) |
| phase A | |
| Sample temperature | 5° C. |
| Injection volume | 60 μL |
| Run time | 50 minutes |
| Run mode | Gradient |

TABLE 8
Gradient Conditions:
Gradient Conditions

| Time (min.) | % Mobile phase B |
|---|---|
| 0.0 | 50 |
| 15.0 | 50 |
| 20.0 | 90 |
| 30.0 | 90 |
| 31.0 | 50 |
| 50.0 | 50 |

TABLE 9
CAD Parameters:
Instrument Parameter Value

| Evaporator temp Low | 35° | C. |
|---|---|---|
| Wait ready | ±5.0K | |
| Power function | 1.0 | |
| Peak width | 0.1 | min. |
| Data collection | 10 | Hz |
| Filter | 5 | Sec. |

CAD detector signal in terms of Baseline was non-fluctuating and stable. Samples were injected in the following sequence to ensure that column and CAD detector was conditioned properly. Negative control (Diluent) was injected to ensure that no peak was eluted at the integration region (mPEG-PAL peak). Six injections of mPEG-PAL standards were injected to ensure stability and reproducibility of the signal/peak at the defined retention time (12.0±1.0 min.). After Standards, Test samples were injected, and signal was recorded. Bracketing standard was injected to ensure reproducibility of area and retention time of the Standard (mPEG-PAL).

TABLE 10
Example Sample Set Sequence Set-up:

| Sequence Line | Sample | No. of Injections |
|---|---|---|
| — | Conditioning | 1 |
| 1 | Negative Control (Diluent) | 1 |
| 2 | mPEG-PAL standard | 6 |
| 3-13 | Samples (up to 10 samples) | 1 |
| 14 | mPEG-PAL standard | 1 |

The data was analyzed with Empower 3. Apex track integration was done for Data integration. The other integration parameters were used for mPEG-PAL standard and test samples. These values were adjusted based on the change in the chromatographic profile of free PEG/mPEG-PAL peak. Processing and reporting were performed on CAD signal/channel.

TABLE 11
Processing Method Conditions:

| Time | | Value | |
|---|---|---|---|
| (min.) | Type | (min.) | Stop time |
| 0.0 | Threshold | 10 | — |
| 0.0 | Peak Width | 80 | — |
| 0.0 | Minimum height | 10 | — |
| 0.0 | Valley to Valley | — | — |
| 0.0 | Inhibit Integration | 7.8 | — |
| 13.0 | Inhibit Integration | — | — |

### System Suitability:

The retention time of PEG-PAL PEG peak should be 12.0±1.0 minutes.
The difference of retention time of the mPEG-PAL peak in bracketing standard should be NMT 1.0 minutes of average of initial six reference standard.
The % RSD of Peak Area of mPEG-PAL peak in 6 reference standard replicates must be ≤10%.
Tailing for first injection of mPEG-PAL standard peak should not be more than 1.6.

### Calculation:

$$Purity of mPEG - Pal = 100 - Water content as per COA or use 20 k fraction ( \% )$$

$$Free PEG in sample ( mg / mL ) = \frac{AT}{AS} \times \frac{SW}{DS} \times \frac{DT}{TW} \times \frac{P}{100}$$

Where,
AT: Area of free PEG in test sample solution
AS: Average area of mPEG-PAL in 6 standard solutions
SW: Standard weight in mg
DS: Dilution of standard in mL
DT: Standard volume taken in mL
TW: Sample volume taken in mL
P: Purity of mPEG-PAL standard

### Results:

TABLE 12
Calculation of mPEG-PAL conc, in PEGylated GCSF.

| | | |
|---|---|---|
| Std weight | 50.16 | mg |
| Std dilution | 1000 | mL |
| Avg std area | 940219309 | pA*s |

| | |
|---|---|
| Purity | 99% |

| Sample type | Batch No. | Peak Area (pA) | mg/mL |
|---|---|---|---|
| Pegylated GCSF | Batch 1 | 142286185 | 0.02 |
| | Batch 2 | 125265046 | 0.01 |
| | Batch 3 | 87906662 | 0.01 |

LOQ: 0.01 mg/mL

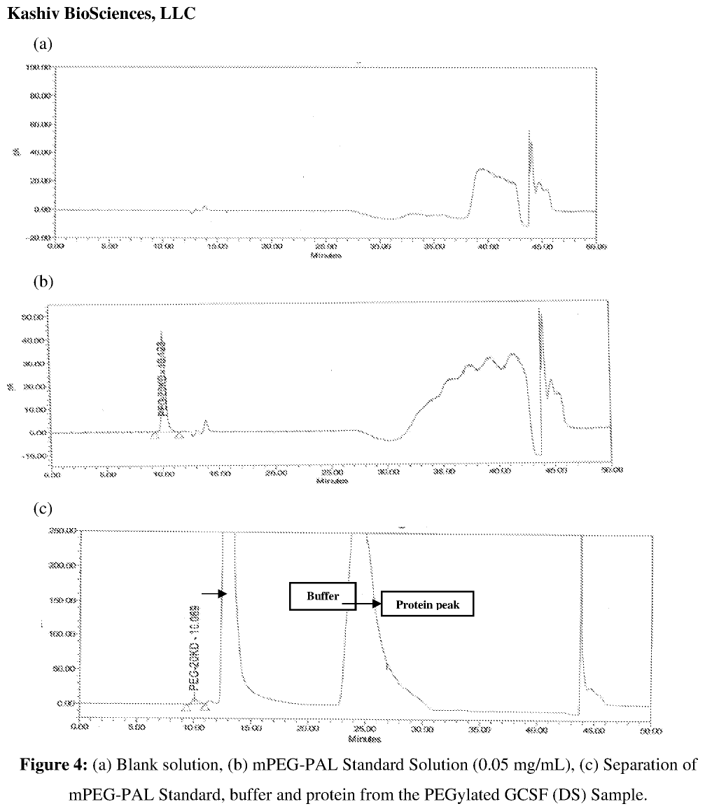

Refer FIGS. 4(a), 4(b) & 4(c) for the chromatogram.

### Example 4: Method Suitability Testing

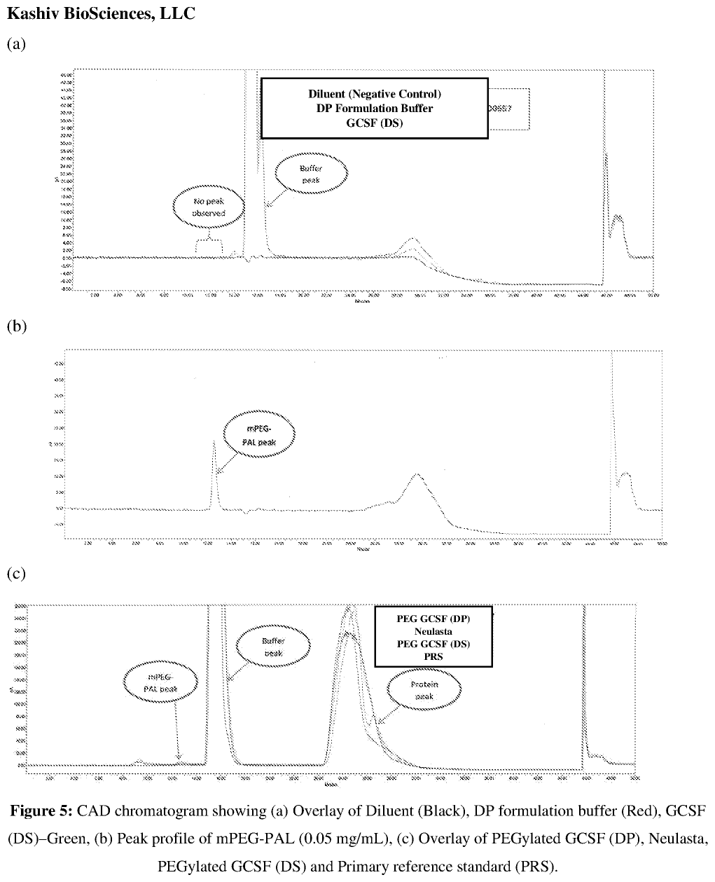

Specificity: Specificity was determined by making a single injection each of Primary reference standard which is pegylated GCSF PEGylated GCSF (DP), PEGylated GCSF (DS), mPEG-PAL (free PEG), formulation excipients used for the preparation of PEGylated GCSF (DP), GCSF and diluent. The CAD chromatograms of the blank preparations (both diluent and PEGylated GCSF-DP formulation buffer) showed no peaks that elute near the retention time of free PEG (FIG. 5a). Thus, the method is specific for the quantification of free PEG in PEGylated GCSF samples in the presence of matrix solution.

TABLE 13(a)
Specificity summary for test samples.

| Validation | | |
|---|---|---|
| Parameter | Sample Name | Results |
| Specificity | Neulasta | Single and well separated peak of free |
| | | PEG was present at 12.8 min. |
| | | between retention time from 11.0 to |
| | | 13.0 minutes. |
| | PEGylated GCSF | Single and Well separated peak of free |
| | (DS) | PEG was present at 12.7 min between. |
| | | retention time from 11.0 to 13.0 |
| | | minutes. Amount of free PEG is less |
| | | than LOQ. |
| | PEGylated GCSF | Single and Well separated peak of |
| | (DP) | free PEG was present at 12.9 min. |
| | | between retention time from 11.0 to |
| | | 13.0 minutes. |
| | Primary Reference | Single and Well separated peak of free |
| | standard (PRS) | PEG was present at 12.9 min. between |
| | | retention time from 11.0 to 13.0 |
| | | minutes. |
| | Formulation | No peak was observed in the |
| | excipients used | formulation buffer in the region of |
| | in PEGylated | 11.0 to 13.0 minutes. |
| | GCSF (DP) | |
| | GCSF (DS) | No peak was observed in the |
| | | formulation buffer in the region of |
| | | 11.0 to 13.0 minutes. |
| | mPEG-PAL (Free | A fully separated (no co-elution of the |
| | PEG) | peak with nearby peaks) and single |
| | | peak was present at 12.6 min. between |
| | | retention time from 11.0 to 13.0 |
| | | minutes for mPEG-PAL. |

### Accuracy:

The accuracy of the method for the mPEG-PAL peak was determined by spiking following concentrations of mPEG-PAL in PEGylated GCSF (11.9 mg/mL): 0.005 mg/mL, 0.010 mg/mL, 0.025 mg/mL, 0.050 mg/mL and 0.075 mg/mL representing 10%, 20%, 50%, 100% and 150%, of the nominal concentration of 0.05 mg/mL for mPEG-PAL test samples. Each level was prepared in triplicate (3 independent preparations) and single injection of each preparation was injected (Table 6 (b)). Percent recovery was calculated by comparing the average experimental concentration determined from the regression parameters calculated in Table 6 (b) with the theoretical concentration at each level.

TABLE 13(b)
mPEG-PAL spiked in PEGylated GCSF primary reference standard data Summary.

| | | mPEG- | | | | | Calculated | |
|---|---|---|---|---|---|---|---|---|
| Level of | Amount | PAL peak | | | | Actual | sample | |
| sample | loaded | Area | Average | Standard | % | Average | Conc. | % |
| (mg/mL) | (μg) | (mAu*s) | area | deviation | RSD | area | (mg/mL) | Recovery |
| Control | NA | 0 | 0 | 0 | NA | NA | NA | NA |
| | | 0 | | | | | | |
| | | 0 | | | | | | |
| 0.005 | 0.3 | 86681642 | 79746355 | 6054190 | 7.6 | 79746355 | 0.005 | 101 |
| | | 75517422 | | | | | | |
| | | 77040001 | | | | | | |
| 0.01 | 0.6 | 149062514 | 134939089.3 | 12232891 | 9.1 | 134939089 | 0.011 | 108 |
| | | 127676709 | | | | | | |
| | | 128078045 | | | | | | |
| 0.025 | 1.5 | 292659281 | 265811563.3 | 24857370 | 9.4 | 265811563 | 0.025 | 98 |
| | | 243596296 | | | | | | |
| | | 261179113 | | | | | | |
| 0.05 | 3 | 533322159 | 493867286 | 41054643 | 8.3 | 493867286 | 0.048 | 97 |
| | | 451380929 | | | | | | |
| | | 496898770 | | | | | | |
| 0.075 | 4.5 | 694946535 | 756810022 | 66341717 | 8.8 | 756810022 | 0.076 | 101 |
| | | 748614982 | | | | | | |
| | | 826868549 | | | | | | |

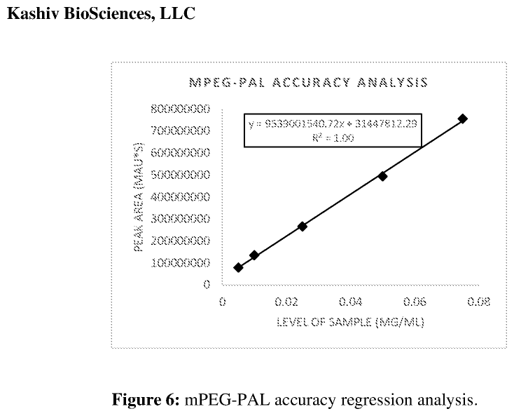

Refer FIG. 6 for mPEG-PAL accuracy regression analysis graph.

The data presented in Table 6 (b) demonstrates that the method is accurate for recovery of the main peak in the concentration range of 0.005-0.075 mg/mL. The calculated percent recoveries for levels fall within 97%-108%.

### Precision:

The precision of the method was determined by Analyst 1 (preparing and analyzing six independent) preparations of PEGylated GCSF PRS spiked with mPEG-PAL standard where the final concentration of mPEG-PAL in protein was 0.05 mg/mL as mentioned in PTL-2261. mPEG-PAL in spiked samples were calculated as mg/mL. Calculated the average, STDV, and % RSD for mPEG-PAL peak area and mg/mL (n=6)

TABLE 13(c)
Precision Data Summary for mPEG-PAL spiked in PEGylated GCSF PRS.

| | Nominal | | Calculated | Average | | |
|---|---|---|---|---|---|---|
| | Conc. | Peak Area | Conc. | Conc. | Standard | % |
| Sample Name | (mg/mL) | (pA) | (mg/mL) | (mg/mL) | deviation | RSD |
| Method precision - 1 | 0.05 | 428812172 | 0.04 | 0.04 | 0.004 | 10 |
| Method precision - 2 | 0.05 | 386502173 | 0.04 | | | |
| Method precision - 3 | 0.05 | 404636267 | 0.04 | | | |
| Method precision - 4 | 0.05 | 345314617 | 0.03 | | | |
| Method precision - 5 | 0.05 | 333209902 | 0.03 | | | |
| Method precision - 6 | 0.05 | 362531959 | 0.03 | | | |

TABLE 13(d)
Precision Data acceptance criteria summary for
mPEG-PAL spiked in PRS.Validation Parameter

| Precision Data acceptance | | |
|---|---|---|
| criteria summary for mPEG- | | |
| PAL spiked in PRS.Validation | | |
| Parameter | Results | Pass/Fail |
| Precision | % RSD (n = 6) for peak | Pass |
| | area of mPEG-PAL peak | |
| | was NMT 3%. | |
| | % RSD of mPEG-PAL | Pass |
| | (mg/mL) in spiked PRS or | |
| | DS, for all the 6 | |
| | preparations (n = 6) was | |
| | NMT 10%. | |

The data presented in Table 6(c) and 6(d) demonstrates that the method is precise for the quantitation of mPEG-PAL (free Polyethylene glycol).

1. A method for separating and detecting free or residual polyethylene glycol in pegylated protein mixture comprising:
(a) obtaining a protein mixture comprising a pegylated protein and at least free or residual polyethylene glycol;
(b) subjecting the protein mixture to at least two chromatography steps;
wherein the first chromatography step is capable to bind the protein of interest and second chromatography step is capable to separate based on molecular weight or size of the analyte;
(c) eluting the protein mixture; and
(d) separating and detecting of free PEG and PEGylated protein in the protein mixture by a suitable detector.

2. The method as claimed in claim 1, wherein the free PEG and PEGylated protein in the protein mixture is further quantified by suitable technique.

3. The method as claimed in claim 1, wherein the PEGylated protein in the protein mixture is PEG-GCSF.

4. The method as claimed in claim 1, wherein the chromatography step comprises at least two chromatography steps.

5. The method as claimed in claim 3, wherein the two chromatography steps are RP-HPLC and SEC-HPLC.

6. The method as claimed in claim 4, wherein the two chromatography steps are RP-HPLC followed by SEC-HPLC connected in a series or performed as an individual chromatography.

7. The method as claimed in claim 1, wherein the elution of protein mixture as a “first protein mixture” eluted in flow through mode and as “second protein mixture” eluted in bind eluate mode from RP-HPLC.

8. The method as claimed in claim 6, wherein the “first protein mixture” is free or residual polyethylene glycol and “second protein mixture” is PEGylated protein.

9. The method as claimed in claim 7, wherein free or residual polyethylene glycol is subjected to SEC-HPLC followed by PEGylated protein.

10. The method as claimed in claim 1 and claim 2, wherein detection or quantification of free or residual PEG and PEGylated protein in the protein mixture by Charged Aerosol Detector.

11. The method as claimed in claim 1, wherein the buffer components are eluted first from the SEC followed by free PEG or residual PEG and PEGylated proteins.

12. A method for detecting or quantifying pegylated protein, free or residual polyethylene glycol in pegylated protein mixture comprising:
a) obtaining a protein mixture comprising a pegylated protein and at least free or residual polyethylene glycol;
b) subjecting the protein mixture to RP-HPLC column to obtain first treated protein mixture in flow through mode and second treated protein mixture binds to RP-HPLC;
c) subjecting the first treated protein mixture to Size exclusion chromatography (SEC) column to obtain quantifiable protein mixture;
d) eluting the second treated protein mixture from the RP-HPLC;
e) quantification and detection of free PEG in the quantifiable protein mixture; and
f) quantification and detection of pegylated protein in the second protein mixture.

13. The method as claimed in claim 12, detection and quantification of free PEG and PEG reagents are performed by Charged Aerosol Detector.

14. The method as claimed in claim 12, elution of the second treated protein mixture from RP-HPLC using mobile phases comprising acetonitrile and ammonium acetate.
